(19) United States
(12) Patent Application Publication
(10) Pub. No.: US 2022/0323270 A1
(43) Pub. Date: Oct. 13, 2022

(54) DENTAL ABSORPTION ROLL DEVICE AND METHOD OF USE

(71) Applicant: Cary Manaois, Chicago, IL (US)

(72) Inventor: Cary Manaois, Chicago, IL (US)

(21) Appl. No.: 17/720,071

(22) Filed: Apr. 13, 2022

Related U.S. Application Data
(60) Provisional application No. 63/174,396, filed on Apr. 13, 2021.

(51) Int. Cl. A61F 13/53; A61F 13/56

(52) U.S. Cl. CPC A61F 13/53; A61F 13/56; A61F 2013/53035; A61F 2013/5666

(57) ABSTRACT

A dental absorption roll device and method of use provides a pair of absorptive rolls that are tethered at both ends by a cord/thread is disclosed. The absorptive rolls are stuffed between one each side of mouth near the teeth being worked on. The absorptive rolls are fabricated from an absorbent material to absorb excess saliva generated during the dental procedure. The absorptive rolls absorb saliva, and maintain space in the mouth for better viewing of the teeth. The cord is forcibly introduced into the space between the teeth; thereby mounting device to teeth. The mounted cord generates tension between the absorption rolls and the teeth. This helps retain the absorption rolls in their respective position on each side of the mouth, one roll on the cheek side, and the other roll next to the tongue. Such secure placement of absorptive rolls allows dental professional to work alone.

### RELATED APPLICATIONS

This application claims priority under 35 U.S.C. 119 of U.S. patent application Ser. No. 63/174,396 filed Apr. 13, 2021, which is hereby incorporated by reference in its entirety.

### TECHNICAL FIELD

This disclosure relates generally to a dental apparatus, and more particularly to an apparatus to keep a dry field around one or more teeth.

### BACKGROUND

Conventionally, cotton rolls are held in place with the fingers of the clinician to keep the space around one or more teeth dry during any kind of operations on the teeth. Typically, an assistant would have to assist as the loose cotton rolls move around.

It is thus desirable to provide an apparatus that eliminates the manual help required by other assistants/technicians to hold the cotton rolls in a particular and also provide absorption capabilities

### SUMMARY

It is to be understood that both the foregoing general description and the following detailed description are exemplary and explanatory only and are not restrictive of the invention, as claimed. The present invention relates generally to an apparatus to keep a dry field around one or more teeth and a method thereof.

The present invention in one aspect discloses an apparatus to keep a dry field around one or more teeth. The apparatus comprises a plurality of rolls defined by an absorbent body. The apparatus comprises a pliable absorptive member connecting each of the plurality of rolls. In an embodiment, the pliable absorptive member comprises a first portion, a second portion, and a third portion. In an embodiment, the first portion of the pliable absorptive member is inserted across the first roll and the second roll such that the inserted first portion of the pliable absorptive member is in a direction perpendicular to a length of the first roll and the second roll. In an embodiment, a portion of the pliable absorptive member inserted from the first roll towards the second roll defines the first portion. In an embodiment, a second portion of the pliable absorptive member is disposed along a length of the second roll. In an embodiment, the third portion of the pliable absorptive member is inserted across the second roll and the first roll such that the inserted third portion of the pliable absorptive member is in a direction perpendicular to the length of the first roll and the second roll. In an embodiment, a portion of the pliable absorptive member inserted from the second roll towards the first roll defines the third portion. In another embodiment the apparatus is constructed by attaching two cords perpendicular to the of the first roll and the second roll.

The present invention discloses another aspect for a method for keeping a dry field around one or more teeth using the disclosed apparatus. The method comprises pulling a pair of absorption cords apart until the cords and a pair of attached absorption rolls are generally perpendicular to each other. The method comprises aligning the cords, such that the absorption roll is in place to be positioned between the gums and the teeth. The method comprises introducing the cords between adjacent spaces between teeth, whereby the device mounts to the teeth. The method comprises adjusting the absorption rolls along the channel that forms between the gums and the cheek to a desired position, such that the cheeks are separated from the teeth, and the teeth are more visible. The method comprises removing the absorptive rolls from between the cheek and the gums, and removing the cords from between the spaces in the teeth.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a perspective view of an exemplary apparatus to keep a dry field around one or more teeth, in accordance with an embodiment of the present invention;

FIG. 2 illustrates the plurality of rolls positioned around teeth in a mouth, in accordance with an embodiment of the present invention;

FIG. 3 illustrates a perspective view of the apparatus, showing a position of a single cord relative to the plurality of rolls, in accordance with an embodiment of the present invention;

FIG. 4 illustrates a flowchart of an exemplary method for using apparatus for keeping a dry field around one or more teeth, in accordance with an embodiment of the present invention.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

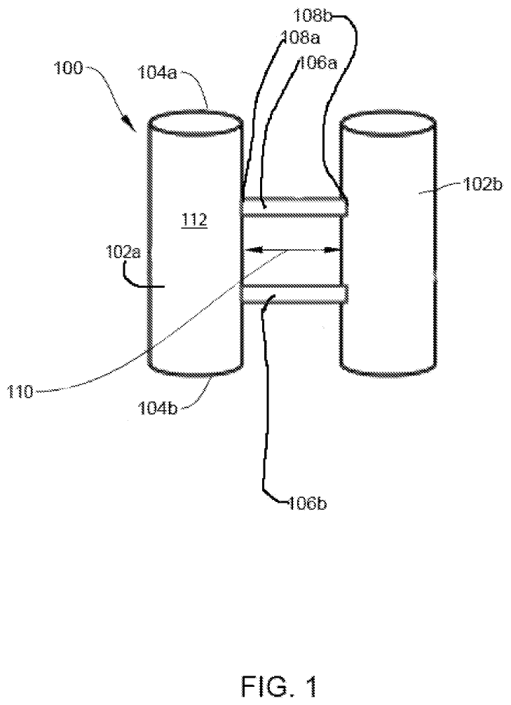

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

At the outset, it should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. § 112.

The term absorptive rolls and plurality of rolls have been used interchangeably and refer to the same component of the apparatus. The term cords and pliable absorptive member have been used interchangeably and refer to the same component of the apparatus.

One objective of the present invention is to provide an apparatus to a dental professional such that no assistance is required from an assistance to hold cotton rolls between the cheeks and the teeth. Another objective of the present invention is to isolate the teeth and keep the teeth dry during dental procedures. In one aspect, the absorptive rolls 102a-b includes at least one of the following materials: cotton, cellulose pulp fibers, and natural fibers.

In another aspect, the cords 106a-b can be non-elastic, rigid, semi-elastic, or elastic material. In another aspect, the absorptive rolls 102a-b comprise a cylindrical shape. Another objective is to enable the dental professional to work alone, without requiring assistance in holding the cotton rolls in place between the gum and the teeth. Yet another objective is to enable successful isolation and placement of dental bonding materials used in a dental procedure, and also allows the dental professional to work independently.

Another objective is to place the rolls on each side of the mouth—one roll on the cheek side, and the other roll next to the tongue. An exemplary objective is to provide an inexpensive way to manufacture a dental absorption roll device.

Additional objectives are to provide an easy-to-use dental absorption roll device. In an embodiment, the apparatus may be commercially named as Dentaboat which is a construction of two cotton rolls attached together with an elastic or non-elastic cord/thread.

The present invention relates generally to a dental absorption roll device and method of use. More so, the dental absorption roll device provides a pair of absorptive rolls that are tethered at both ends by a cord; whereby the cord is forcibly introduced into the space between the teeth, on each side of the mouth—one roll on the cheek side, and the other roll next to the tongue in order to mount the device to the teeth and generate tension between teeth and absorptive rolls to minimize movement and displacement of absorptive rolls from between the cheeks and the gum.

A dental absorption roll device and method of use provides a pair of absorptive rolls that are tethered at both ends by a cord. The absorptive rolls are stuffed between one each side of mouth near the teeth being worked on. The absorptive rolls are fabricated from an absorbent material to absorb excess saliva generated during the dental procedure, and also absorb liquid compositions introduced into the mouth during the dental procedure. The absorptive rolls absorb saliva, and maintain space in the mouth for better viewing of the teeth. The cord is forcibly introduced into the space between the teeth; thereby mounting device to teeth. The mounted cord generates tension between the absorption rolls and the teeth. This helps retain the absorption rolls in their respective position on each side of the mouth, one roll on the cheek side, and the other roll next to the tongue. This secure placement of absorptive rolls allows dental professional to work alone. Absorptive rolls serve the dual purpose of absorbing saliva, and maintaining space between the cheeks and gums for better viewing of the teeth.

Cords, which are joined to both absorptive rolls, are forcibly introduced into the space between the teeth; thereby mounting the entire device to the teeth. In this manner, mounting the cords to the teeth generates tension between absorptive rolls and the teeth. Tension serves to better tether absorptive rolls, helping retain absorptive rolls in their respective position on each side of the mouth, one roll on the cheek side, and the other roll next to the tongue. This secure placement of absorptive rolls allows the dental professional to work alone, without requiring assistance in holding the rolls in place between the gum and the teeth.

FIG. 1 illustrates a perspective view of an exemplary apparatus to keep a dry field around one or more teeth dental, in accordance with an embodiment of the present invention.

As shown in FIG. 1, dental absorption roll device 100 (also termed as apparatus 100) comprises a pair of absorptive rolls 102a-b (termed as plurality of rolls) that work together inside the mouth of a patient being operated on in the dental procedure.

In one embodiment, pair of absorptive rolls 102a-b have substantially the same shape and dimensions and are defined by an absorbent cylindrical body 112 having a pair of free ends 104a-b. Such absorptive materials that comprise absorbent body 112 may include, without limitation, cotton, cellulose pulp fibers, and natural fibers. In one embodiment, absorptive rolls 102a-b are between 3″ to 7″ in length. However, device 100 is scalable, such that smaller or larger dimensions may be used. For example, if used with animals, the rolls have a larger diameter and length.

In operation, absorptive rolls 102a-b arranged in a generally spaced-apart, parallel relationship. However, absorptive rolls 102a-b have enough flexibility to be malleable and adjusted around the curvature of the teeth. Absorptive rolls 102a-b can also align in a nonparallel relationship with each other, depending on their position in the mouth. However, in most operational procedures, absorptive rolls 102a-b are substantially parallel while placed in the mouth between the gums and the cheeks. For example, the apparatus 100 (i.e., dental absorption roll device 100) to keep a dry field around one or more teeth comprises a plurality of rolls (i.e., absorptive rolls 102a-b) that are defined by an absorbent body. Each of the plurality of rolls are made at least one of cotton, cellulose pulp fibers, and natural fibers. In an embodiment, each of the plurality of rolls is cylindrical in shape. In an embodiment, each of the plurality of rolls is configured to absorb saliva within the mouth and around the one or more teeth and other fluids that may be dispensed within the mouth while performing the dental procedures.

In an embodiment, in an open position, each of the plurality of rolls are pulled in an outward direction in the open position, the each of the plurality of rolls are placed on either side of one or more tooth, wherein the plurality of rolls fit between gums and opposing sides of the one or more tooth. In an embodiment, in a closed position, each of the plurality of rolls are disposed adjacent to each other.

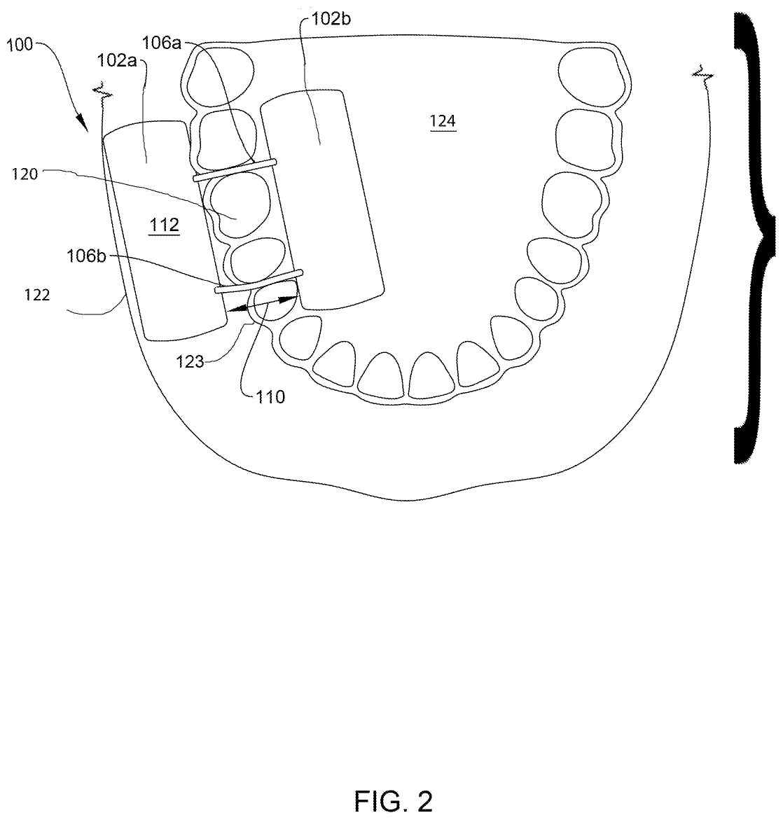

FIG. 2 illustrates the plurality of rolls (i.e., absorptive rolls 102a-b) positioned around teeth and adjacent to a cheek having two separate threads, in accordance with an embodiment of the present invention. In essence, absorptive rolls 102a-b fit between the teeth and cheek on opposing sides of the teeth. Specifically, the rolls are introduced on each side of the teeth 120, i.e. one roll adjacent the cheek 122 and gum line 123, and the other roll inside the mouth 124. The dental procedure may include a simple tooth examination, or a more complex root canal. Any dental procedure that requires the cheek to be separated from the gums and teeth so as to enable the dental professional to view the teeth more clearly can benefit from device 100.

Referring to FIG. 2, device 100 includes a pair of cords 106a-b (106a is the first portion and 106b is the second portion) that serve to tether the absorptive rolls 102a-b in the aforementioned configuration. Cords 106a-b are defined by a pair of cord ends 108a-b that fixedly join with the absorptive rolls 102a-b, and specifically near the free ends 104a-b of the absorptive rolls 102a-b. However, cords 106a-b can also be placed closer to the center of the absorptive rolls 102a-b in some embodiments. In one embodiment, cord ends 108a-b are integral to the body of the absorptive rolls 102a-b.

In one embodiment, cords 106a-b are elastic in nature, allowing stretching and increased manipulative positions for cords 106a-b and the absorptive rolls 102a-b. Due to the elastic configuration of the cords 106a-b, absorptive rolls 102a-b may be pulled apart, twisted, and otherwise manipulated for better fit into the mouth. In one possible embodiment, cords 106a-b are arranged in a spaced-apart relationship with each other while holding the absorptive rolls 102a-b. In another embodiment, cords 106a-b are arranged in a perpendicular relationship with the absorptive rolls 102a-b. However, as absorptive rolls 102a-b are manipulated in the mouth more acute or obtuse angles between cords 106a-b and absorptive rolls 102a-b may occur.

Cords 106a-b are configured to fit into the space between teeth. Thus, the cords 106a-b also serve to mount device 100 into the mouth by squeezing between the tight space between teeth. This fixed mounting configuration of cords 106a-b serves as a tether to generate tension (shown as arrow 110) between cords 106a-b while placed between the gum and the cheek. This tethering configuration helps minimize movement of absorptive rolls 102a-b while positioned between the gums and the teeth. The tension also keeps absorptive rolls 102a-b in place so that the dental professional does not need a second pair of hands to hold absorptive rolls 102a-b in place. Cords 106a-b, in essence, do this job. This frees the hands of the dental professional to work solely on the dental procedure, and negates the need for an assistant.

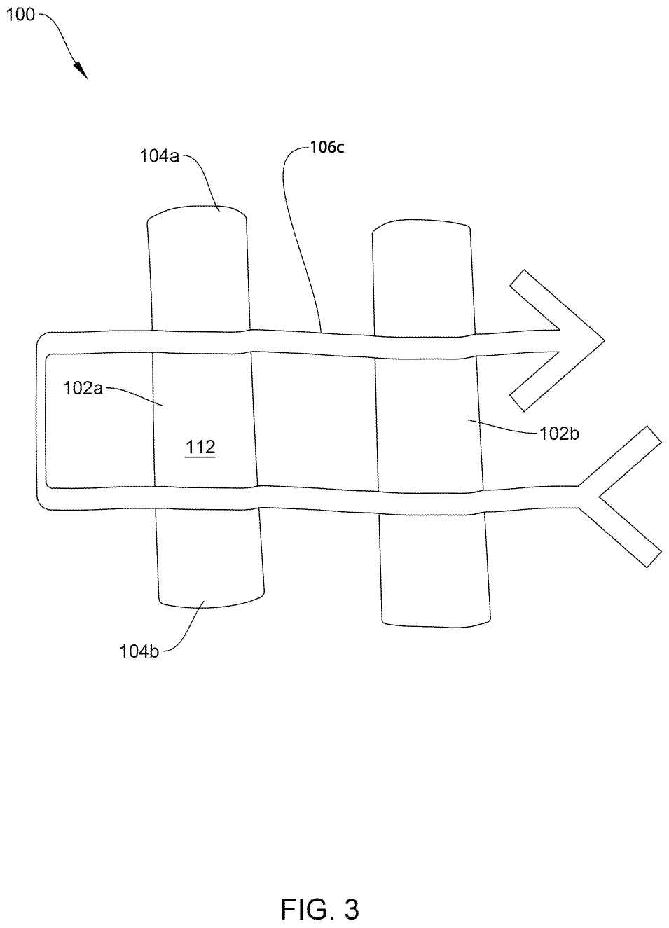

FIG. 3 illustrates a perspective view of the apparatus, showing a position of a single cord/thread relative to the plurality of rolls, in accordance with an embodiment of the present invention. FIG. 3 illustrates the plurality of rolls (i.e., absorptive rolls 102a-b) having one continuous thread 106c, in accordance with an embodiment of the present invention. The apparatus 100 (i.e., dental absorption roll device 100) further comprises a pliable absorptive member connecting each of the plurality of rolls. Continuous thread 106c is preferably comprised of an elastic material but it may be comprised of a non-elastic thread. The pliable absorptive member is configured to restrict mobility of each of the plurality of rolls when placed on either side of one or more tooth. In an embodiment, the pliable absorptive member connecting each of the plurality of rolls fits into a space between teeth, whereby the elastic cord generates tension between the plurality of rolls for minimizing movement from between gums and teeth.

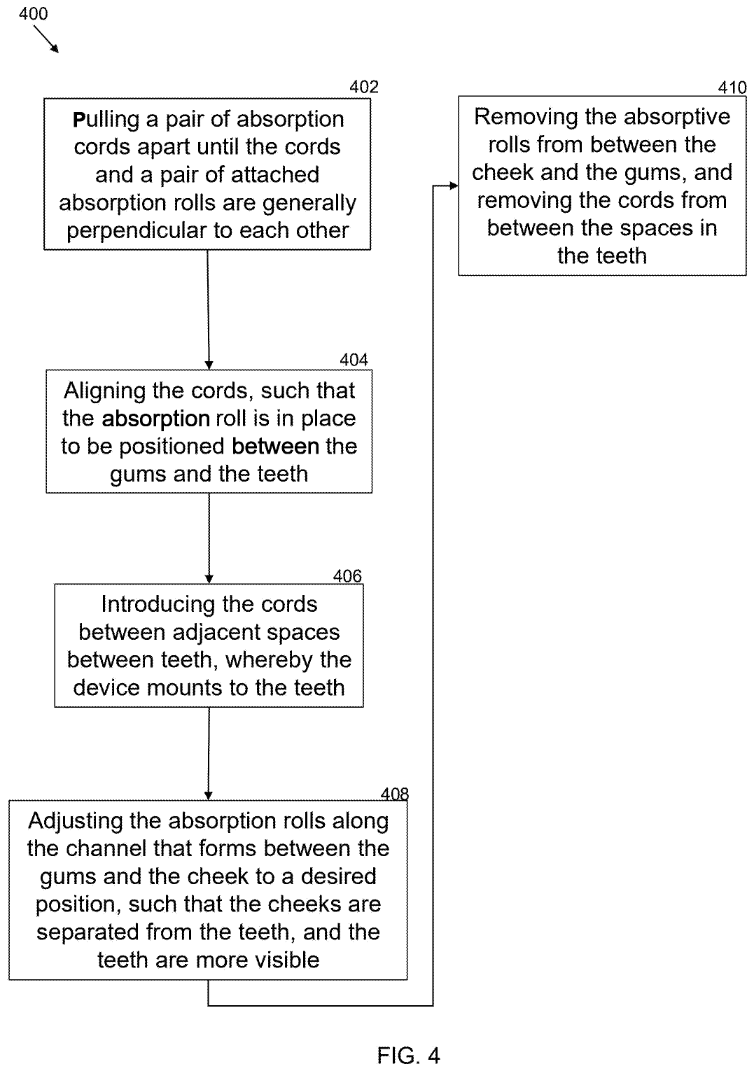

FIG. 4 illustrates a flowchart of an exemplary method 400 for using apparatus (i.e., dental absorption roll device 100) for keeping a dry field around one or more teeth, in accordance with an embodiment of the present invention.

Method 400 may include an initial step 402 of pulling a pair of absorption cords apart until the cords and a pair of attached absorptive rolls 102a-b are generally perpendicular to each other. A gentle tugging motion is sufficient force to prepare the device for introduction into the mouth. Another step 404 comprises aligning the cords, such that the absorption roll is in place to be positioned between the gums and the teeth. This may include holding the cords above the teeth that are to be worked on.

Yet another step 406 includes introducing the cords between adjacent spaces between teeth, whereby the device mounts to the teeth. The cords are pulled apart and forcibly squeeze between the space. As the absorptive rolls 102a-b fit on each side of the mouth, one roll on the cheek side, and the other roll next to the tongue. The device is mounted to the teeth and a tension is generated between the absorptive rolls 102a-b and the teeth. This helps retain the absorptive rolls 102a-b in a fixed position during the dental procedure.

Yet another step 408 includes adjusting the absorptive rolls along the channel that forms on each side of the mouth, one roll on the cheek side, and the other roll next to the tongue, such that the cheeks are separated from the teeth and the teeth are more visible. At this point, the absorptive rolls 102a-b begin to absorb the lever and moisture from the mouth. Also, the dental professional does not require assistance in separating the cheek from the gums. A final step 410 may include removing the absorptive rolls 102a-b from between the cheek and the gums, and removing the cords from between the spaces in the teeth. This step 410 may be performed after the dental procedure has been completed.

Various embodiments of the invention provide method and an apparatus for keeping a dry field around one or more teeth while performing dental procedures. The claimed apparatus isolates the teeth and keeps it dry so that the dental professional can work simultaneously on other things. By implementation of the claimed limitations, the clinician does not need anyone to assist in holding loose cotton rolls in place. Successful isolation and placement of dental bonding materials adds to the longevity filling material used and allows the operator to work independently.

The claimed steps as discussed above are not routine, conventional, or well understood in the art, as the claimed steps enable the following solutions to the existing problems in conventional technologies.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

It is intended that the disclosure and examples be considered as exemplary only, with a true scope and spirit of disclosed embodiments being indicated by the following claims. In some embodiments, any combination of shapes, sizes, and textures of rolls and the pliable flexible member may be used.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. An apparatus to keep a dry field around one or more teeth, the apparatus comprising:
a first roll and a second of roll each defined by an absorbent cylindrical body;
a first cord and a second cord connecting the first roll to the second roll, wherein a first end of the first cord is connected to the first roll and a second end of the first cord is connected to the second roll such that the first cord is in a direction perpendicular to a length of the first roll and the second roll, and wherein a first end of the second cord is connected to the second roll and a second end of the second cord is connected to the first roll such that the second cord is in a direction perpendicular to a length of the first roll and the second roll.

2. The apparatus of claim 1, wherein the first roll and the second roll each have a first end a second end, and wherein the first end of the first cord is connected to the first end of the first roll and a second end of the first cord is connected to the first end of the second roll, and wherein a first end of the second cord is connected to the first end of the second roll and a second end of the second cord is connected to the second end of the first roll.

3. The apparatus of claim 1, wherein the cords connecting the first roll and the second of roll are elastic.

4. The apparatus of claim 3, wherein the first cord and second cord are parallel to each other and the first roll and the second of roll are of an equal length.

5. The apparatus of claim 1, wherein the cords connecting each of the plurality of rolls fit into a space between teeth.

6. The apparatus of claim 1, wherein each of the plurality of rolls are made at least one of cotton, cellulose pulp fibers, and natural fibers.

7. The apparatus of claim 1, wherein each of the first and the second rolls is cylindrical in shape.

8. The apparatus of claim 1, wherein in a closed position, each of the plurality of rolls are disposed adjacent to each other.

9. The apparatus of claim 1, wherein the first and the second cord is a single continuous elastic member.

10. The apparatus of claim 1, wherein each of the first and the second rolls is configured to absorb saliva within the mouth and around the one or more teeth.

11. A method for keeping a dry field around one or more teeth, the method comprising:
pulling a pair of absorption cords apart until the cords and a pair of attached absorption rolls are generally perpendicular to each other;
aligning the cords, such that the absorption roll is in place to be positioned between the gums and the teeth;
introducing the cords between adjacent spaces between teeth, whereby the device mounts to the teeth;
adjusting the absorption rolls along the channel that forms between the gums and the cheek to a desired position, such that the cheeks are separated from the teeth, and the teeth are more visible; and
removing the absorptive rolls from between the cheek and the gums, and removing the cords from between the spaces in the teeth.
